(19) United States
(12) Patent Application Publication
(10) Pub. No.: US 2005/0131268 A1
(43) Pub. Date: Jun. 16, 2005

(54) System and method for delivering a therapeutic agent for bone disease

(76) Inventor: Karen D. Talmadge, Los Altos Hills, CA (US)

Correspondence Address: FISH & RICHARDSON P.C., PO BOX 1022, MINNEAPOLIS, MN, 55440-1022, US

(21) Appl. No.: 10/990,285

(22) Filed: Nov. 15, 2004

Related U.S. Application Data
(63) Continuation-in-part of application No. 10/265,922, filed on Oct. 7, 2002.
Application No. 10/265,922 is a continuation-in-part of application No. 10/044,843, filed on Jan. 11, 2002, now abandoned.
Application No. 10/044,843 is a continuation-in-part of application No. 10/054,736, filed on Oct. 24, 2001, now abandoned.
Application No. 10/054,736 is a continuation-in-part of application No. 08/986,876, filed on Dec. 8, 1997, now abandoned.
Application No. 08/986,876 is a continuation-in-part of application No. 08/911,805, filed on Aug. 15, 1997, now abandoned.
Application No. 08/911,805 is a continuation-in-part of application No. 08/871,114, filed on Jun. 9, 1997, now Pat. No. 6,248,110.
Application No. 08/871,114 is a continuation-in-part of application No. 08/659,678, filed on Jun. 5, 1996, now Pat. No. 5,827,289.
Application No. 08/659,678 is a continuation-in-part of application No. 08/485,394, filed on Jun. 7, 1995, now abandoned.

(51) Int. Cl. A61N 5/00

(52) U.S. Cl. 600/3

(57) ABSTRACT

Various systems, and methods are provided for transporting a therapeutic agent to the interior of a skeletal support structure. In one implementation, an expandable structure has at least one lumen configured to receive a radiation source, and is configured for insertion into a skeletal support structure. The expandable structure is further configured to create a void within the skeletal support structure. Furthermore, the expandable structure comprises at least one lumen configured to contain a radiation source.

This application is a continuation-in-part of and claims priority to co-pending U.S. patent application Ser. No. 10/265,922, filed Oct. 7, 2002, which is a continuation-in-part of U.S. patent application Ser. No. 10/044,843 filed Jan. 11, 2002, now abandoned, which is a continuation-in-part of U.S. patent application Ser. No. 10/054,736, filed Oct. 24, 2001, now abandoned, which is a continuation-in-part of U.S. patent application Ser. No. 08/986,876 filed Dec. 8, 1997, now abandoned, which is a continuation-in-part of U.S. patent application Ser. No. 08/911,805, filed Aug. 15, 1997, now abandoned, which is a continuation-in-part of U.S. patent application Ser. No. 08/871,114 filed Jun. 9, 1997, now issued U.S. Pat. No. 6,248,110, which is a continuation-in-part of U.S. patent application Ser. No. 08/659,678 filed Jun. 5, 1996, now issued U.S. Pat. No. 5,827,289, which is a continuation-in-part of U.S. patent application Ser. No. 08/485,394, filed Jun. 7, 1995, now abandoned, the disclosures of which are herein incorporated in their entirety by reference thereto.

### TECHNICAL FIELD

This invention relates to therapy for bone disease. In particular, this invention relates to systems and methods for accessing bone to deposit a therapeutic agent.

### BACKGROUND

When cancellous bone becomes diseased, for example, because of osteoporosis, avascular necrosis or cancer, it can no longer provide proper support to the surrounding cortical bone. The bone therefore becomes more prone to compression fracture or collapse.

Radiation therapy and chemotherapy are commonly used to treat cancerous conditions, such as spinal metastases. Radiation therapy can be administered in any of a number of ways, including external-beam radiation, stereotactic radiosurgery, and permanent or temporary interstitial brachytherapy.

### SUMMARY

The methods and devices described allow for depositing a therapeutic agent directly to the interior volume of a skeletal support structure such as a vertebral body while minimizing exposure of surrounding tissue to radiation and to harmful side effects of the therapy. In one embodiment, an expandable structure has at least one lumen configured to receive a radiation source, and is configured for insertion into a skeletal support structure. The expandable structure is further configured to create a void within the skeletal support structure. Furthermore, the expandable structure comprises at least one lumen configured to contain a radiation source.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows an apparatus including a first elongated member and a second elongated member having an expandable structure.

FIG. 2A shows an expandable structure connected to a second elongated member in an unexpanded configuration.

FIG. 2B shows an expandable structure connected to a second elongated member in an expanded configuration.

FIG. 2C is cross-sectional end view of the expandable structure of FIG. 2B.

FIG. 2D is a cross-sectional end view of the second elongated member of FIG. 2A.

FIG. 3 shows an apparatus including a first elongated member and a second elongated member having an expandable structure, disposed within a vertebral body.

FIG. 4A shows an expandable structure including a primary lumen and a number of secondary lumens, in an unexpanded configuration.

FIG. 4B shows an expandable structure of FIG. 4A in an expanded configuration.

FIG. 4C is cross-sectional end view of the expandable structure of FIG. 4B in an expanded configuration.

FIG. 4D is a cross-sectional end view of the second elongated member of FIG. 4A.

Like reference symbols in the various drawings indicate like elements.

### DETAILED DESCRIPTION

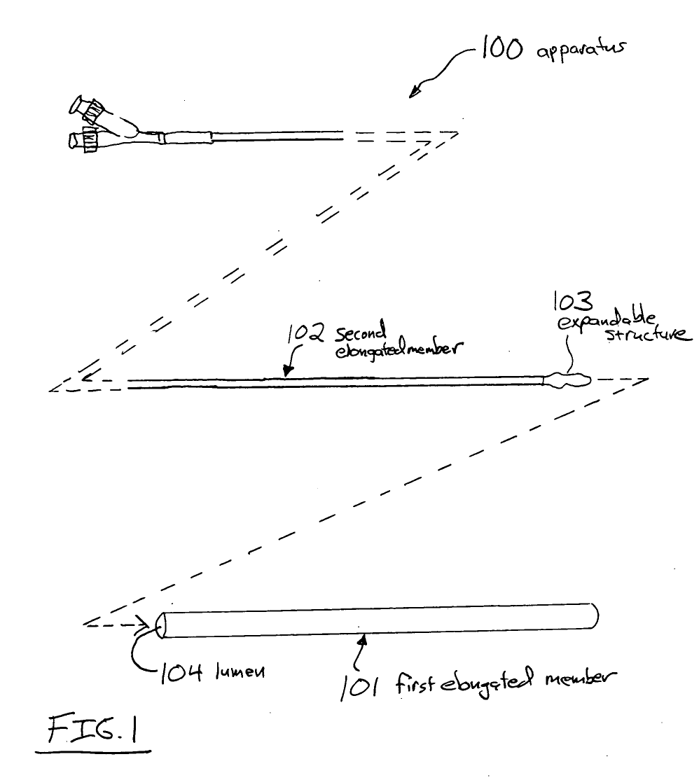

FIG. 1 shows an apparatus 100 for providing a radiation source to the interior of a skeletal support structure. The apparatus 100 includes a first elongated member 101 having a lumen 104, wherein the first elongated member 101 is configured to provide non-axial access to the interior of the skeletal support structure. In one implementation, the first elongated member 101 can be configured, for example, as a cannula, catheter, needle, trocar or other suitable access device. The apparatus 100 includes a second elongated member 102 configured to transport a therapeutic agent through the lumen 104 of the first elongated member 101 to the interior of the skeletal support structure. Finally, the apparatus 100 includes an expandable structure 103 configured for insertion into a skeletal support structure, wherein the expandable structure 103 is configured to create/expand a void within the skeletal support structure.

The therapeutic agent transported to the interior of the skeletal support structure can include but is not limited to, for example a chemotherapeutic agent, a radiation source or combinations thereof.

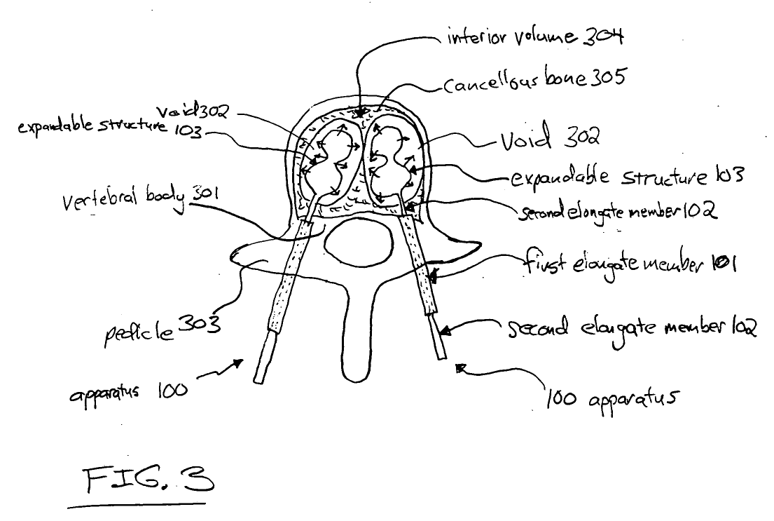

The skeletal support structure accessed using the apparatus 100 can include, but is not limited to, for example, bone, cartilage and ossified derivatives thereof, membrane bone and cartilage bone. As shown in FIG. 3, in one implementation the first elongated member 101 facilitates access to a skeletal support structure consisting of a vertebral body 301. Particularly, as shown in FIG. 3, the interior volume 304, typically containing cancellous bone 305, is accessed via the first elongated member 101 through the pedicle 303 of the vertebral body 301. Access to the interior of the vertebral body 301 can be accomplished via the side walls of the vertebral body 301, for example, using an extrapedicular, postero-lateral, lateral, or anterior approach; or also via the vertebral body 301 endplates.

As shown in FIGS. 1, 2A-2C and 3, in one implementation the second elongated member 102 has a distal end that includes an expandable structure 103 configured to create a void 302 within the skeletal support structure (e.g., see void 302 in FIG. 3). As used herein, “expandable” refers to a property of the structure that includes elastic, non-elastic, and partially elastic/non-elastic expansion. The expandable structure can be made from a deformable plastic or metal material. As used herein, “create a void” is meant to include both expanding an existing void in a skeletal support structure in addition to expanding the interior of a skeletal support structure to produce a void. It is contemplated that a skeletal support structure accessed with the apparatus 100 can comprise a void prior to being accessed or upon being accessed. It is further contemplated that such a prior existing or contemporaneously formed void can be further expanded using the above-described expandable structure 103.

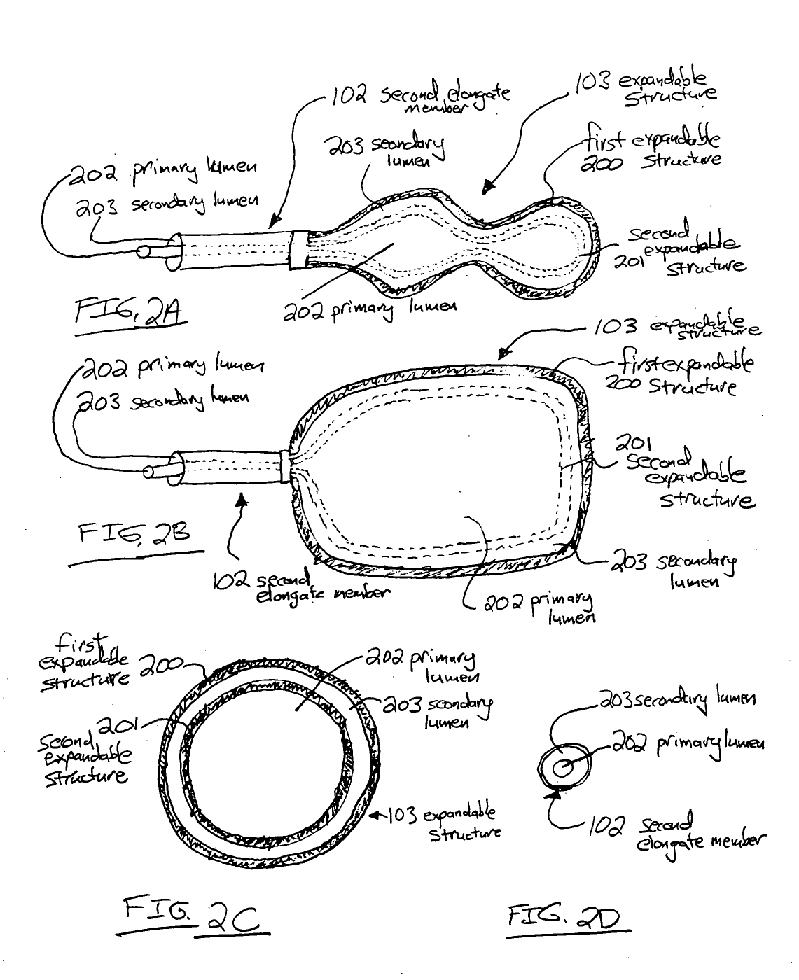

As shown in FIGS. 2A-D, the expandable structure 103 is coupled to the second elongated member 102 and can be configured to deliver a radiation dose to the interior of the skeletal support structure. In the implementation shown, the expandable structure 103 is comprised of a first expandable structure 200 and a second expandable structure 201. The first and second expandable structures 200 and 201 are configured to define a primary lumen 202 and a secondary lumen 203 (see FIGS. 2A-C). Examples of suitable primary and secondary lumens 202 and 203 include but are not limited to, inner open spaces or cavities within a tube, sleeve, pocket, pouch, sac, bag, or vessel. In one implementation, the primary and secondary lumens 202 and 203 extend into and span, substantially the length of the second elongated member 102. A radiation source can be received between the first and second expandable structures 200 and 201 in the secondary lumen 203. Expansion of the expandable structure 103 can be controlled by the addition of a substance (e.g., a fluid) to, for example, the primary lumen 202.

In one embodiment, as shown in FIGS. 2B and 2C, the first expandable structure 200 and second expandable structure 201 are configured to achieve a correlated expanded state. As used herein, “correlated expanded state” is meant to describe an expansion of the first and second expandable structures 200 and 201, synchronously or asynchronously, at a same or different rates or any suitable manner to achieve a fixed relationship or a variable one depending on the elasticity or other properties of the expandable structures 200 and 201. For example, the first expandable structure 200 can be expanded to a first size while coincidentally the second expandable structure 201 can be expanded to substantially the same first size. Alternatively, the first expandable structure 200 can be expanded to a first size while coincidentally the second expandable structure 201 can be expanded to a second size. After expansion, the first and second expandable structures 200 and 201 are configured to unexpand. The unexpanded configuration can facilitate, for example, removal of part or all of the apparatus 100 from a skeletal support structure.

In one implementation, the second elongated member 102 is configured at the distal end to be remotely visualized (e.g., using fluoroscopy, X-ray, MRI, CT scan, or computer-aided imaging) while the distal end is inside the interior of the skeletal support structure. Such a configuration can include suitable marking means for remote visualization disposed substantially near the distal end of the second elongated member 102 (not shown). For example, such a configuration can be accomplished using one or more radiopaque marker bands. In another example, the expandable structure 103 can be comprised of one or more radiopacifer. Examples of radiopacifiers include but are not limited to, iodine (such as CONRAY® available from Mallinckrodt), gadoliminum, tungsten, tantalum, barium, strontium. It is contemplated that a radiopacifier or a radiopaque substance can be disposed within the primary lumen 202, and/or the secondary lumen 203 of the expandable structure 103. Alternatively, the first expandable structure 200 and/or the second expandable structure 201 can be co-manufactured with a radiopacifier, or could be coated on the inside or outside of the expandable structure 103.

In another implementation, the first elongated member 101 is further comprised of a means for penetrating a skeletal support structure (not shown). As used herein, “means for penetrating a skeletal support structure” include, but are not limited to, a stylet, drill, trocar, needle assembly, catheter and any other practicable device for penetrating a skeletal support structure. The means for penetrating a skeletal support structure can be coupled to the distal end of the first elongated member 101, or configured for use in conjunction with the elongated member 101. The means for penetrating a skeletal support structure can also be coupled to the second elongated member 102.

In one implementation, the radiation source can be positioned at a predetermined location (e.g., in relation to the skeletal support structure). Where step-wise positioning is indicated, the predetermined location can be a series of dwell positions (discussed below) substantially near or within a skeletal support structure. The positioning of the radiation source can be controlled by the configuration of the first and second elongated members 101 and 102. The relevant configuration of the first and second elongated members 101 and 102 can include but is not limited to, for example, indexes or markings that communicate the positional relationship between the first and second elongated members 101 and 102 (not shown). Positioning of the radiation source can be aided by the use of CT scan to determine the measurement of the skeletal support structure and to calculate the distance to place in the desired position. Positioning the radiation source within the skeletal support structure can also be controlled based on the expansion of the expandable structure 103. For example, the relative amount of expansion of the expandable structure can provide positioning of the radiation source deployed within the expandable structure 103 at a number of predetermined locations within or near the skeletal support structure (e.g., within the interior volume 304 of a vertebral body 301).

In one implementation, the radiation source is configured to provide a dose of radiation substantially localized within the interior of the skeletal support structure. In particular, the form and substance of the radiation source, in conjunction with the configuration and deployment of the second elongated member 102, can be adjusted to provide a desired localized dose. Such a dose is calculable. For example, a dosimetry plan can be used to calculate how long the radiation source should spend (dwell time) in specified localized positions (dwell position) within the skeletal support structure.

In a particular implementation, the radiation source is received within the void 302 using an afterloader (not shown). The afterloader can be coupled to a lumen of the second elongated member 102 of the apparatus 100 for introduction of a radiation source within the lumen.

In one embodiment, the radiation source is a radionuclide. The radionuclide can be in the form of a liquid, seed, needle, pellet, particle, microsphere, or any other suitable form of radionuclide for radiation treatment. The radionuclide can be comprised of Au-198, Co-60, Cs-137, I-125, I-135, Ir-192, P-32, Pd-103, Ra-226, Rh-106, Ru-106, Sr-90, Y-90 or any other isotope suitable for radiation treatment, and can be liquid, solid or generated in situ by electronic brachytherapy (available from Xoft, Inc.)

In one implementation, the apparatus 100 is further comprised of a radiation shield configured to shield the radiation source (not shown). The shield can be configured to contain emissions from the radiation source until release of the emissions is desired, for example, to provide dose to local bone. In one embodiment, the shield can be configured to enclose the second elongated member 102 and/or the expandable structure 103 coupled thereto (not shown). In another embodiment, the shield can be comprised of a metal mesh, which is inserted into the interior of the skeletal support structure after expansion of the expandable structure 103. In yet another embodiment, the shield can be incorporated into the first expandable structure 200 and/or second expandable structure 201.

Referring to FIGS. 1 and 3, a method of using the apparatus 100 described above comprises: inserting non-axially, into an interior of a skeletal support structure, a first elongated member 101 having a lumen 104 that defines an access path into the interior of the skeletal support structure; inserting, into the lumen 104, a second elongated member 102 configured to transport a radiation source to the interior of the skeletal support structure; and transporting the radiation source through the lumen 104 into the interior of the skeletal support structure.

In one implementation, the method of using the apparatus 100 further comprises expanding at least a portion of the second elongated member 102 to create a void 302 in the interior of the skeletal support structure. Expanding the second elongated member 102 to create a void 302 can optionally be executed before or after transporting the radiation source through the lumen.

In another implementation, the method of using the apparatus 100 further comprises depositing a supportive material in the void 302. The supportive material can be bone cement (e.g., polymethyl methacrylate (PMMA), ceramics), human bone graft (autograft and allograft), synthetic derived bone substitutes such as calcium sulfate, calcium phosphate and hydroxyapatite. Additionally, in another implementation, the supportive material can include a chemotherapeutic agent.

In one implementation, the method step of transporting the radiation source further comprises placing the radiation source at one or more dwell positions. Furthermore, the method can include determining multiple dwell positions to provide a dose of radiation substantially localized within the interior of the skeletal support structure. Determining dwell positions includes but is not limited to computer software determination of dwell positions.

In another implementation, the method step of inserting the first elongated device 101 comprises inserting the first elongated device 101 into an interior volume 304 of a vertebral body 301 through a pedicle 303 of a vertebral body 301 (see FIG. 3). In another implementation, two or more first elongated devices 101 are inserted through one or more pedicles 303 of a vertebral body 301. Alternatively, the step of inserting the first elongated device 101 can comprise inserting one or more of the first elongated device 101 into an interior volume 304 of a vertebral body 301 through the side walls of the vertebral body 301. For example, the first elongated device 101 can be inserted by way of an extrapedicular, postero-lateral, lateral, or anterior approach; or alternatively via the vertebral body 301 endplates. In another implementation, the method step of inserting the first elongated device 101 comprises inserting the first elongated device 101 into a skeletal support structure including bone, cartilage and ossified derivatives thereof, membrane bone and cartilage bone.

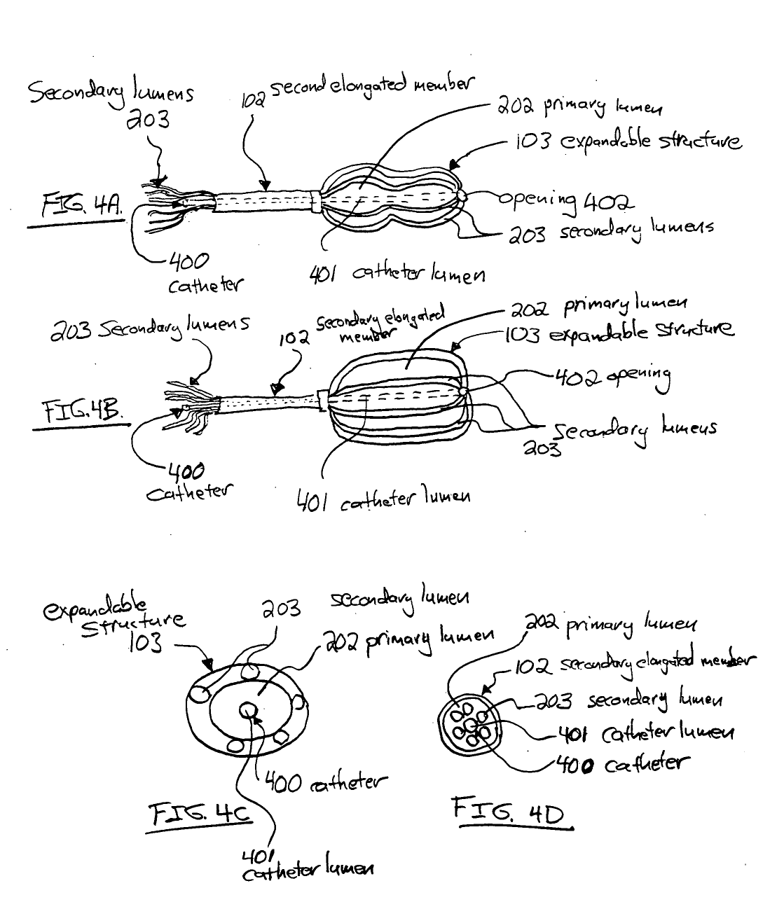

As described above, an apparatus 100 is provided for use within the interior of a skeletal support structure that includes an expandable structure that can be comprised of at least one lumen configured to contain a radiation source. As shown in FIG. 4A-D, the apparatus 100 can comprise an expandable structure 103 configured for insertion into a skeletal support structure and optionally configured to be coupled to an elongated member such as the second elongated member 102 described above (see FIGS. 1, 2A, 2B and 2D).

The expandable structure 103 can include a first layer and -a second layer. The first and second layers can be configured to receive a radiation source between the first layer and the second layer. In one implementation, as shown in FIGS. 4A-D, the expandable structure 103 includes a primary lumen 202, and at least one secondary lumen 203. The primary and secondary lumens 202 and 203 are configured to be in fluid communication with a lumen in the second elongated member 102. In the implementation shown in FIGS. 4A-D, the primary and secondary lumens 202 and 203 extend into and substantially span the length of the second elongated member 102.

In another implementation, the first and second expandable structures 200 and 201 are comprised of a material having properties or characteristics including compliant or non-compliant and combinations thereof. As used herein, “compliant” includes the properties of flexibility in an elastic, expandable or bendable way. Additionally, as used herein, “non-compliant” includes a rigid quality although, depending on the context, may not imply complete rigidity. By varying the incorporation of such material when manufacturing the first and second expandable structures 200 and 201, different sizes, shapes and consistencies of the un-expanded and expanded first and second expandable structures 200 and 201 can be achieved.

The expandable structure 103 can be configured for insertion into a skeletal structure in an unexpanded configuration. Insertion of the unexpanded expandable structure 103 into the skeletal support structure can create a void 302 within the skeletal support structure. The expandable structure 103 can further be configured to create or expand a void within a skeletal support structure upon expansion of the expandable structure 103. As shown in FIGS. 3, 4A and 4B, in one implementation, the expandable structure 103 is configured for insertion into a void (e.g., void 302) in an unexpanded configuration (see FIG. 4A), expanded into the void to an expanded configuration (see FIG. 4B), and unexpanded after expansion. A void 302 in the interior volume 304 of a vertebral body 301 can be increased by the expansion of the first and second expandable structures 200 and 201 within the interior volume 304. As shown in FIG. 3, the void 302 is provided when one or more of the first and second expandable structures 200 and 201 are expanded, which result from the compaction of cancellous bone 305.

In one implementation, the expandable structure 103 is comprised of an expandable material having an interior lumen, the expandable material being configured to expand to a first shape, and to reversibly expand to a second shape. As shown in FIGS. 2A-D, the expandable structure 103 of the apparatus 100 can comprise a first expandable structure 200, and a second expandable structure 201 disposed within the first expandable structure 200. Additionally, the first and second expandable structures 200 and 201 include interior and exterior walls. A substance (e.g., a fluid) can be introduced into the interior lumen (shown in FIGS. 2A-C as primary lumen 202), where the fluid can be caused to force against the interior wall of the second expandable structure 201 to provide expansion of the expandable structure 103 including the first and second expandable structures 200 and 201 (not shown). As shown in FIGS. 2A-C, there is a secondary lumen 203 disposed between the first and second expandable structures 200 and 201. In one implementation, the secondary lumen 203 is configured to receive a substance (e.g., a fluid). Particularly, the substance received in the secondary lumen 203 can be a radiation source.

In one implementation, the first and second expandable structures 200 and 201 are configured for optional removal of either the first, second or both expandable structures 200 and 201 from the distal end of the second elongated member 102 of the apparatus 100 (not shown). For example, the first expandable structure 200 can be configured for removal from the apparatus 100, wherein upon removal, the second expandable structure 201 remains attached to the second elongated member 102 of the apparatus 100. Alternatively, the second expandable structure 201 can be configured for removal from the apparatus 100, wherein upon removal of the second expandable structure 201, the first expandable structure 200 is left attached to the second elongated member 102 of the apparatus 100. In one implementation, the configuration of the expandable structure 103 forms a tube, sleeve, pocket, pouch, sac, bag, vessel or other suitable enclosed space.

In another implementation, the expandable structure 103 includes an expandable geometry configured to reversibly expand to a desired shape (not shown). Examples of such expandable geometries include, but are not limited to, coiled wires, various types of springs, and self-expanding stents or scaffolds. In one implementation, the apparatus 100 further comprises an insertion sleeve configured to substantially surround the expandable structure 103 (not shown). The insertion sleeve can be used to protect the expandable structure during placement and removal from the interior of the skeletal support structure. The insertion sleeve can also protect the surrounding tissues during removal of diseased tissue or instruments, which have come into contact with diseased tissue. The insertion sleeve can help prevent “seeding” of the diseased tissue to other tissues in the access pathway.

As shown in FIGS. 4A and 4B, the expandable structure 103 is comprised of a primary lumen 202 and a secondary lumen 203. The primary lumen 202 can be configured for expanding the expandable structure 103 and the secondary lumen 203 can be configured for containing the radiation source (see FIGS. 4A and 4B). Alternatively, in another implementation, the primary lumen 202 can be configured for containing the radiation source while the secondary lumen 203 is configured for expanding the expandable structure 103.

As shown in FIGS. 4A and 4B, at least one secondary lumen 203 is disposed substantially parallel to the long-axis of the second elongated member 102. Additionally, the at least one secondary lumen 203 is disposed circumferentially about the expandable structure 103 (see FIGS. 4A and 4B). Any of a number of configurations for disposing the at least one secondary lumen 203 in relation to the second elongated member 102 and the expandable structure 103 can be used. For example the number and orientation of the secondary lumens (i.e., the at least one secondary lumen 203) can be varied to optimize containing and transporting the radiation source.

In one implementation, the expandable structure 103 is comprised of one or more lumens having been co-manufactured with the radiation source. For example, the at least one secondary lumen 203 can be co-manufactured with the radiation source. Alternatively, the primary lumen 202 can be co-manufactured with the radiation source.

As shown in FIGS. 4A-D, in one implementation, the at least one lumen 203 of the expandable structure 103 comprises a catheter 400 disposed within and having an opening 402 substantially near the distal end of the expandable structure 103. The opening 402 may accommodate the passage of a guidewire or stiffening stylet. The catheter 400 comprises a catheter lumen 401 and can extend into and substantially span the length of the second elongated member 102 (see FIGS. 4A-D). In another implementation, the at least one lumen 203 of the expandable structure 103 does not include an opening or exit near the distal end of the expandable structure 103.

A method of using the above described expandable structure 103 comprises inserting the expandable structure 103 into a skeletal support structure, wherein the expandable structure 103 comprises at least one lumen configured to receive a radiation source; expanding the expandable structure 103; and transporting the radiation source through the at least one lumen into the skeletal support structure. In another implementation, the method further comprises depositing a supportive material in the skeletal support structure. The supportive material can be bone cement (e.g., polymethyl methacrylate (PMMA), ceramics), human bone graft (autograft and allograft), synthetic derived bone substitutes such as calcium sulfate, calcium phosphate and hydroxyapatite. Additionally, in another implementation, the supportive material can include a chemotherapeutic agent or a radioactive agent.

In one implementation, the apparatus 100, including the expandable structure 103, is configured to provide minimally invasive insertion into a skeletal support structure. For example, as shown in FIG. 3, the apparatus 100 comprising first and second elongated members 101 and 102 includes a minimally invasive configuration for deployment within the interior volume 304 of a vertebral body 301. The configuration is minimally invasive since only a small access through the skin and muscle layers to a desired part of the vertebral body 301 (e.g., through a pedicle 303 as shown in FIG. 3) is required to introduce the apparatus 100 within the interior volume 304. Alternatively, the minimally invasive approach can be applied to the side walls of the vertebral body 301. For example, a desired part of the vertebral body 301 can be accessed through an extrapedicular, postero-lateral, lateral, or anterior approach; or alternatively via the vertebral body 301 endplates. Other skeletal support structures the minimally invasive insertion approach can be applied to include but are not limited to, bone, cartilage and ossified derivatives thereof, membrane bone and cartilage bone.

A number of embodiments of the invention have been described. Nevertheless, it will be understood that various modifications may be made without departing from the spirit and scope of the invention. Accordingly, other embodiments are within the scope of the following claims.

1. An apparatus comprising an expandable structure having at least one lumen configured to receive a radiation source, the expandable structure configured for insertion into a skeletal support structure, and further configured to create a void within the skeletal support structure and comprising at least one lumen configured to contain a radiation source.

2. The apparatus of claim 1, wherein the expandable structure is configured to be coupled to an elongated member for inserting the expandable structure into the skeletal support structure.

3. The apparatus of claim 2, wherein the at least one lumen is disposed substantially parallel to the elongated member.

4. The apparatus of claim 2, wherein the elongated member comprises a first elongated member and a second elongated member, wherein the second elongated member is coupled to the expandable structure, and the first elongated member is configured to receive the expandable structure and the second elongated member.

5. The apparatus of claim 4, further comprising a means for penetrating a skeletal support structure configured to receive the first elongated member.

6. The apparatus of claim 1, wherein the expandable structure includes a primary lumen, and wherein the at least one lumen includes a secondary lumen.

7. The apparatus of claim 1, wherein the at least one lumen is configured to be in fluid communication with a lumen in the elongated member.

8. The apparatus of claim 1, wherein the expandable structure comprises:
a first layer, and
a second layer configured to receive a radiation source between the first layer and the second layer.

9. The apparatus of claim 1, wherein the expandable structure is configured to be inserted into a void in an unexpanded configuration, expanded in the void to an expanded configuration, and unexpanded after expansion.

10. The apparatus of claim 1, wherein the expandable structure is comprised of an expandable material having an interior lumen, the expandable material being configured to expand to a first shape, and to reversibly expand to a second shape.

11. The apparatus of claim 1, wherein the expandable structure is comprised of a folding geometry structure configured to reversibly expand to a desired shape.

12. The apparatus of claim 1, wherein the expandable structure is configured to expand a void within an interior volume of a vertebral body.

13. The apparatus of claim 1, further comprising an insertion sleeve configured to substantially surround the expandable structure.

14. The apparatus of claim 1, wherein two or more lumens are disposed circumferentially about the expandable structure.

15. The apparatus of claim 1, wherein the expandable structure comprises:
a first lumen; and
a second lumen, wherein the first lumen is configured for expansion of the expandable structure, and the second lumen is configured for containing a radiation source.

16. The apparatus of claim 15, wherein the first lumen and second lumen are configured to receive a radiopaque substance therebetween.

17. The apparatus of claim 1, wherein the expandable structure is comprised of one or more lumens having walls, the walls having been co-manufactured with the radiation source.

18. The apparatus of claim 1, wherein the expandable structure is comprised of one or more lumens having walls, the walls having been co-manufactured with a radiopacifier.

19. The apparatus of claim 1, further including the radiation source, and wherein the at least one lumen is configured to contain the radiation source.

20. The apparatus of claim 1, wherein the expandable structure comprises a distal end, and the at least one lumen comprises a catheter having an opening substantially near the distal end.

21. The apparatus of claim 1, wherein the radiation source is comprised of a radionuclide.

22. The apparatus of claim 21, wherein the radionuclide is in a form selected from the group consisting of liquid, wire, seed, needle, pellet, particle and microsphere.

23. The apparatus of claim 21, wherein the radionuclide is selected from the group consisting of Au-198, Co-60, Cs-137, I-125, I-135, Ir-192, P-32, Pd-103, Ra-226, Rh-106, Ru-106, Sr-90 and Y-90.

24. A method comprising:
inserting into a skeletal support structure an expandable structure configured to be coupled to an elongated member, the expandable structure comprising at least one lumen configured to receive a radiation source;
expanding the expandable structure; and
transporting the radiation source through the at least one lumen into the skeletal support structure.

25. The method of claim 24, further comprising:
depositing a supportive material in the skeletal support structure.

26. The method of claim 25, wherein the supportive material is selected from the group consisting of bone cement, human bone autograft, human bone allograft, calcium sulfate, calcium phosphate and hydroxyapatite.
